(19) United States
(12) Patent Application Publication
(10) Pub. No.: US 2004/0043385 A1
(43) Pub. Date: Mar. 4, 2004

(54) Microarray biochip

(76) Inventors: Yo-Hsin Su, Kaohsiung Hsien (TW); Hsi-Lien Lu, Kaohsiung (TW); Hsun-Min Lung, Fengshan City (TW); Chung-Ho Tung, Fengshan City (TW); Ta-Chang Liu, Kaohsiung (TW); Su-Ming Hsu, Kaohsiung (TW); Chih-Kung Lee, Taipei (TW); Shiming Lin, Taipei (TW); Long-Sun Huang, Taipei (TW); Chii-Wann Lin, Taipei (TW); Shih-Yuan Lee, Taipei (TW)

Correspondence Address: BIRCH STEWART KOLASCH & BIRCH, PO BOX 747, FALLS CHURCH, VA, 22040-0747, US

(21) Appl. No.: 10/232,710

(22) Filed: Sep. 3, 2002

(51) Int. Cl. C12Q 1/68; C12M 1/34

(52) U.S. Cl. 435/6; 435/287.2

(57) ABSTRACT

A microarray biochip includes a transparent substrate and a plurality of indented reaction zones formed on the substrate. Each reaction zone has a plurality of adhesive units of a preset height formed thereon in an array fashion, and a top surface to form a first adhesive surface and lateral side surfaces to form second adhesive surfaces. The invention can effectively hold sample solution and retain end label products after calorimetric or fluorometric reaction, and increases reaction area and be adopted for use in optical or reflective detection devices.

### FIELD OF THE INVENTION

The present invention relates to a microarray biochip and particularly to a biochip for use in the detection of nucleic acids, proteins or other biological molecules.

### BACKGROUND OF THE INVENTION

Biochips mainly are used based on specificity existed between biological molecules. Biological probes (mainly include nucleic acids, proteins, carbonhydrates, cells, tissues or the likes) are immobilized on a specific substrate, then biological sample solution (such as blood, urine, body fluid, or saliva) to be tested is applied and mixed on the biochip. The targets in the sample solution can react with the biological probes on the substrate to form the target-probe complex. Then a signal transducer or a detection device may be used to detect the target-probe complex and to process optical, piezoelectric or electrochemical quantitation or image analyses to obtain biological analysis information of the testing samples.

At present there are generally two types of substrates for the immobilization of biological probes on the biochip: (a) one adopts opaque substrates such as polymer films (such as Nylon films) or glass slides evaporating with metal films; (b) another one adopts transparent substrates such as blank glass slides. However, they still have problems when put to practical use, notably:

1. in the (a) type substrates, the metal film (such as gold film) with biological compatibility can form a covalent bonding with a linker or with the probe, and opaque substrates such as Nylon film may also form a covalent bonding through chemical or physical methods (such as Ultraviolet cross-linking). However, these substrates do not allow light to pass through, transmittant scanners such as calorimetric or fluorometric types, or microscopes cannot be used for detection. Only reflective detection devices can be used. The restriction of detection device applicability limits adaptability of some low-cost detection techniques usually used in common laboratory;

2. the (b) type substrates are transparent and can be used in optical detecting devices such as transmittant or reflective scanners or microscopes for doing image analysis and comparison. However, it is often restricted by immobilization method used and its lower immobilization capacity. Also, it requires pre-preparation steps and washing could cause probe leakage and result in errors in qualitative and quantitative analyses;

3. the biochips made by the methods of (a) and (b) types cannot effectively hold end products that retain mark information during marking and testing operations. For instance, when applying on the ELISA testing techniques used in biochips, the chromophore or fluorescent products obtained after enzymatic catalysis are difficult to stay on the substrate when the surface of the biochip are washed or the biochip is moved for detection. As a result, the reading of test results tend to incur errors.

### SUMMARY OF THE INVENTION

Therefore the primary object of the invention is to resolve aforesaid disadvantages. The invention provides a microarray biochip that can firmly and securely hold probes, and has great adaptability for detection devices, and can effectively carry and retain end products.

In order to achieve the foregoing objects, the microarray biochip of the invention includes a transparent substrate which has at least one indented reaction zone. In the reaction zone, there are a plurality of solid or hollow adhesive units. Each solid or hollow adhesive unit has a preset height and affinity to biological molecules, and a top surface and lateral surfaces to form respectively a first adhesive surface and second adhesive surfaces for the immobilization of probes. Thereby the reaction zone may hold sample solution and keep the end mark products. The second adhesive surfaces may provide the probes with the immobilization area for use in transmittant detection. Through the transparent substrate and the spaced adhesive units, it can be adopted for use in optical transmittant or reflective detection device and in calorimetric or fluorometric type of experiments.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a plain view of the invention.

FIG. 2-A is a schematic view of the invention.

FIG. 2-B is a fragmentary enlarged view of FIG. 2-A.

FIG. 3 is a schematic view of the invention in use.

FIG. 4-A is a schematic view of another embodiment of the invention.

FIG. 4-B is a cross section of FIG. 4-A.

FIG. 4-C is a fragmentary enlarged view of FIG. 4-B.

FIG. 4-D is a schematic view of catalytic reaction according to FIG. 4-C.

FIG. 5 is a schematic view of an embodiment of the invention.

### DESCRIPTION OF THE PREFERRED EMBODIMENT

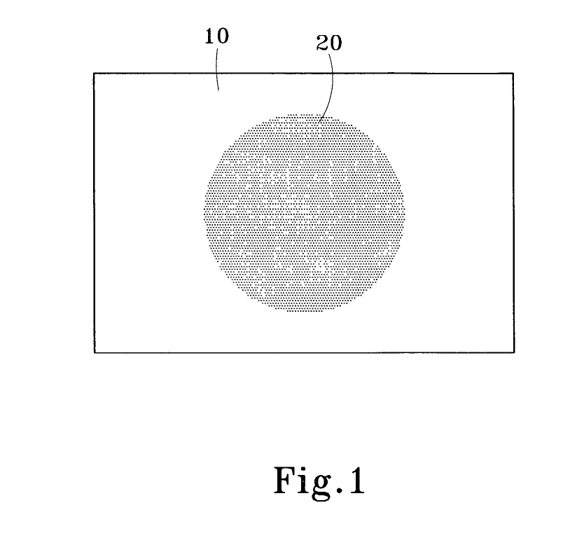

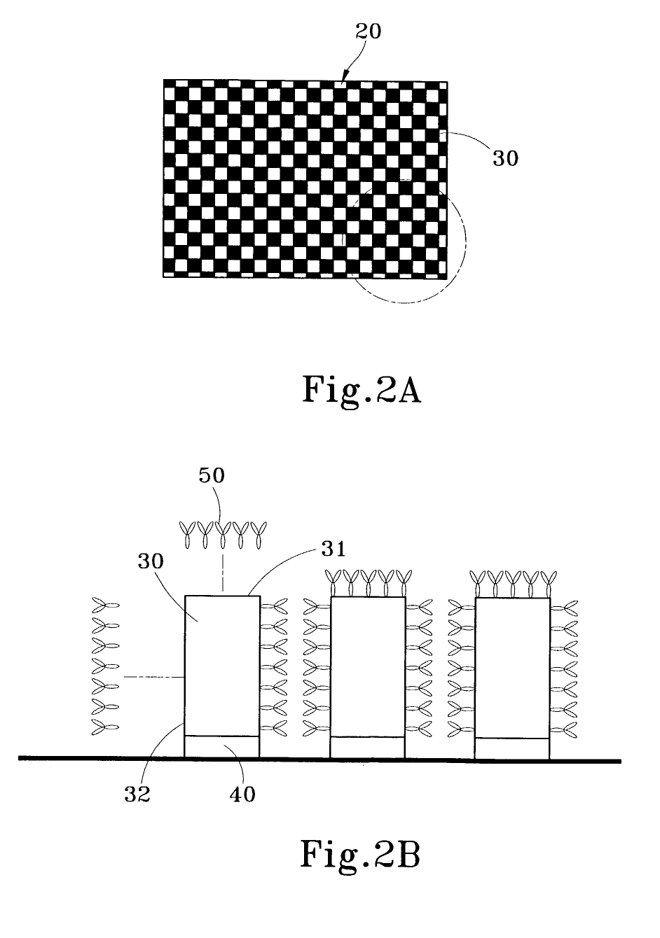

Referring to FIGS. 1, 2-A and 2-B, the microarray biochip of the invention aims at employing specificity between biological molecules to immobilize selected biological molecules on a substrate 10 to form biological probes 50 to interact with corresponding target molecules 61 in test sample solution 60 to generate signals to facilitate readings and analysis for detection device. The microarray biochip of the invention includes a transparent substrate 10 which contains at least one reaction zone 20. In the reaction zone 20, there are a plurality of solid or hollow adhesive units 30. Each solid or hollow adhesive units 30 has a preset height, and a top surface and lateral side surfaces to form respectively a first adhesive surface 31 and second adhesive surfaces 32. The second adhesive surfaces 32 may provide the probes 51 with the immobilization area for use in optical transmittant detection. The gaps between the adhesive units 30 can effectively retain end label products 72 after enzyme 70-catalyted reaction, and through the transparent substrate 10 the biochip allow transmittant type or reflective type of detection device to process reading and analysis of signals.

The substrate 10 is made from glass or other transparent materials. The reaction zone 20 may be formed on the substrate 10 in a matrix configuration, and may be formed in circular, rectangular, or other geometric shapes. Moreover, the adhesive units 30 are made of materials with a great biological compatibility, such as gold, silver, nickel, silicon or copper, preferably gold. The adhesive units 30 may be solid and have a cross section in a shape of a circular, a rectangular or a selected geometric shape. The adhesive units 30 may be hollow and have a cross section with an outer shape of a circular, a rectangular or a selected geometric shape, and with an inner shape of a circular, a rectangular or a selected geometric shape.

During manufacturing, the adhesive unit 30 and an interface layer 40 between the adhesive unit 30 and the substrate 10 may be formed by employing Micro Electro Mechanical System (MEMS) or nanolithography techniques. The adhesive units 30 are laid and distributed in a spaced and matrix fashion with a selected interval formed therebetween on the reaction zone 20. The spaced interval may be 0.1, 1, 10 or 100 ì m, preferably the smaller ones for increasing the area of the first and the second adhesive surfaces. The adhesive units 30 has a preset height of at least 10 nm to allow the lateral second adhesive surfaces 32 having sufficient immobilization surface for biological molecules. The height may be 10, 50, 100, 500 or 1000 nm, preferably smaller than to the interval of the adhesive units 30.

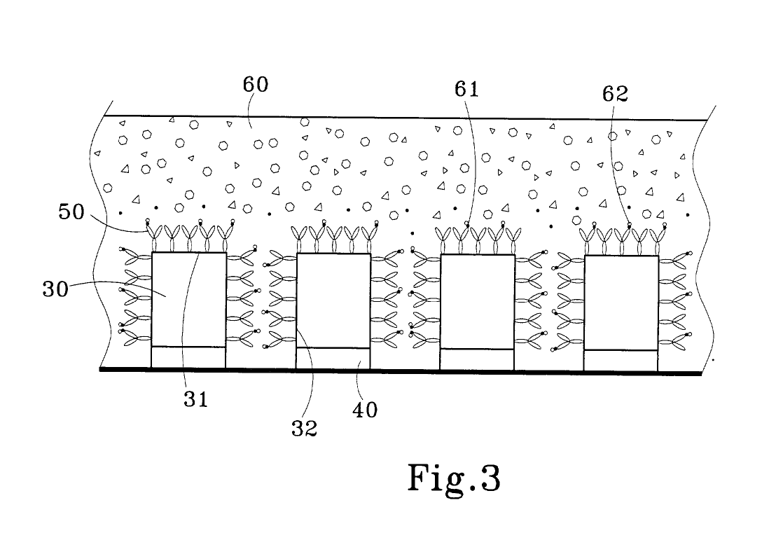

Referring to FIG. 3, when the invention is in use, apply sample solution 60 on the reaction zone 20. The sample solution 60 contains target molecules 61 which conjugates with molecular label 62 (such as fluorescent material fluororecine). The target molecules 61 then start interaction with biological probes 50 which are immobilized to the first and second adhering surfaces 31 and 32. And the molecular label 62 conjugated to the target 61 can generate signal information. The signal information on the first adhesive surface 31 and the second adhesive 32 may be respectively fed to the optical reflective and transmittant type of detection devices to process reading and analysis operations.

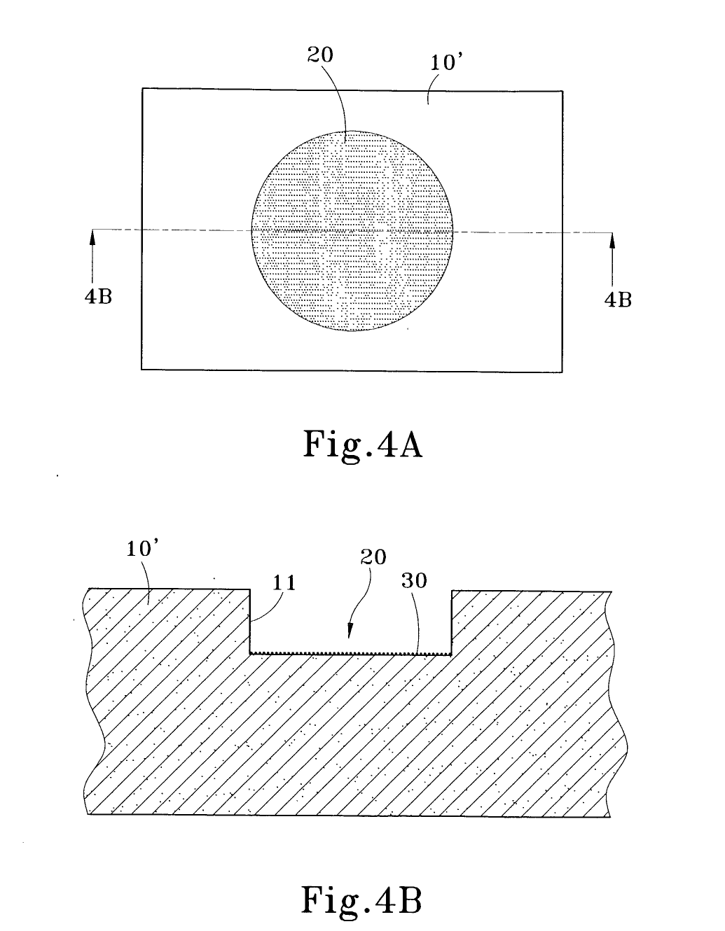

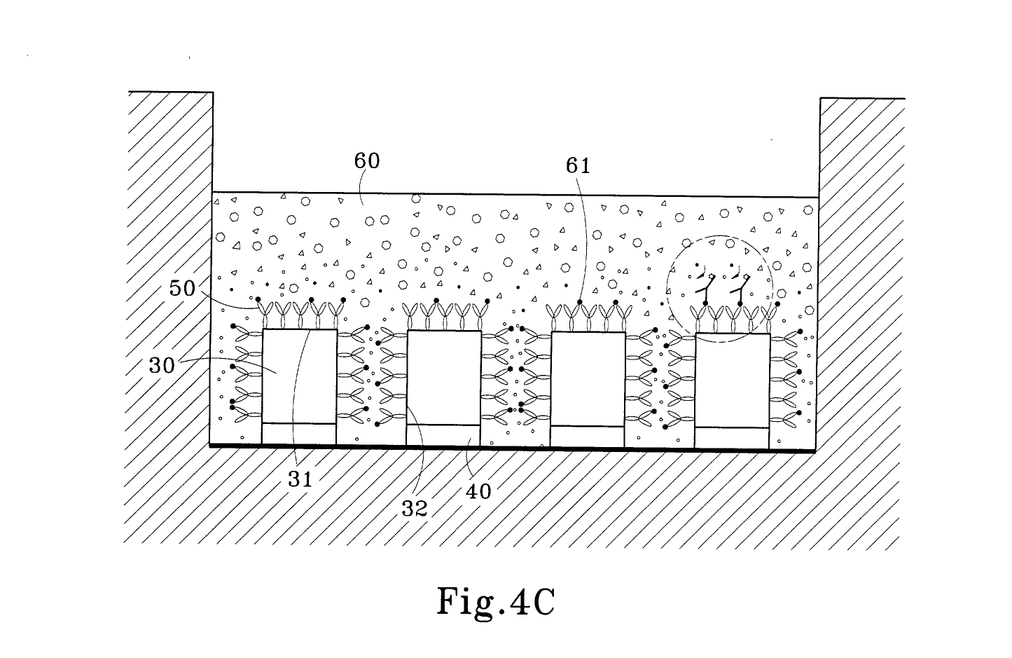

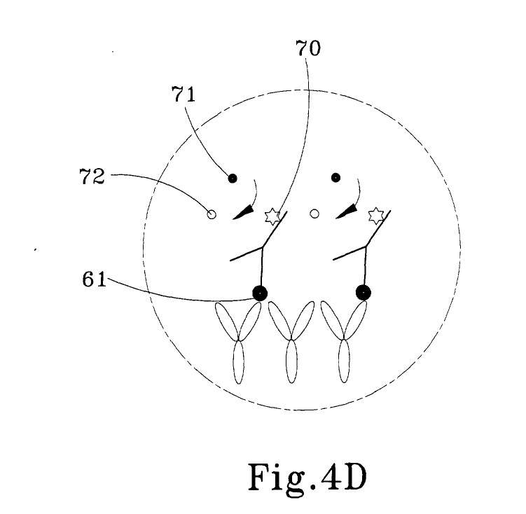

Refer to FIGS. 4-A, 4-B, 4-C and 4-D for another embodiment of the invention, with FIG. 4-A showing a schematic view, FIG. 4-B showing a cross section, FIG. 4-C showing a fragmentary enlarged view, and FIG. 4-D showing a catalytic reaction. The reaction zone 20 is an indented recess formed on the substrate 10′ with a fall distance 11 for holding sample solution 60. The fall distance 11 may prevent the biological probes 50 from damaging when the sample solution 60 is withdrawn. In addition, when applying Enzyme-linked Immunosorbent Assay (ELISA) techniques, substrate 71 that has been catalyzed by enzyme 70 may generate end product 72 with colorimetric or fluorescent property, and the end product 72 may be retained in the fall distance 11. Thereby the end product 72 may be retained without losing when the biochip is moved for detection, and test results can be properly obtained without being affected.

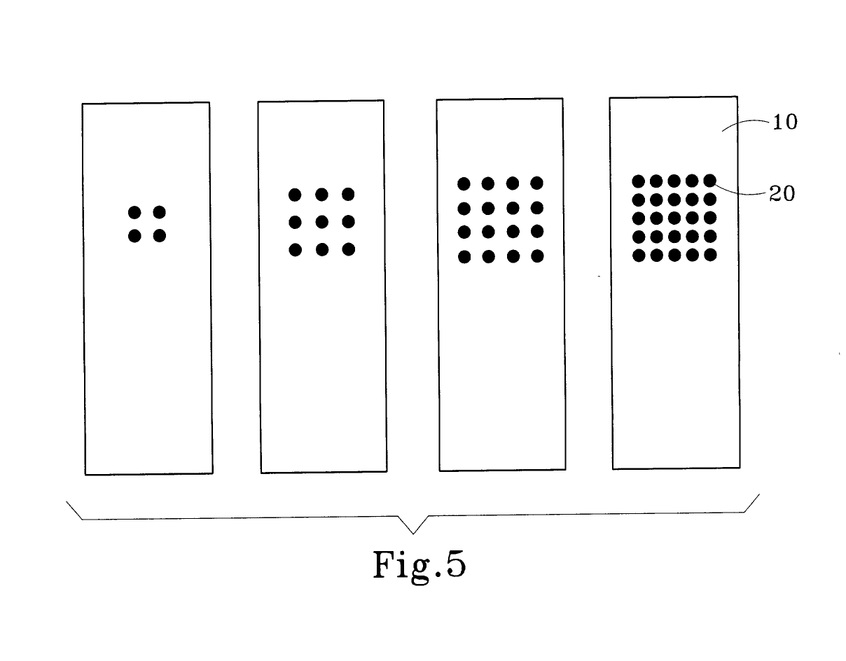

Refer to FIG. 5 for an embodiment of the invention. The reaction zones 20 on the substrate 10 may be configured in a matrix fashion by 2×2, 3×3, 4×4, or 5×5 patterns. The intervals between the reaction zones 20 may be designed and set to match existing arrayer or sample-loading apparatus.

What is claimed is:

1. A microarray biochip employing biological specificity to immobilize selected biological molecules on substrates to form biological probes to interact with corresponding target molecules of test sample solution to generate signals to facilitate reading and analysis for detection apparatus, comprising:
a transparent substrate having at least one reaction zone; and
a plurality of adhesive units fixedly located in the reaction zone, each adhesive unit having a preset height and a top surface to form a first adhesive surface and lateral side surfaces to form second adhesive surfaces for the biological probes to immobilize thereon;
wherein the second adhesive surfaces provide the probes with the immobilization area for use in optical transmittant detection, and the intervals between the adhesive units effectively retaining end label products after colorimetric or fluorometric reaction, and the transparent substrate allowing optical transmittant or reflective detection device to process reading and analysis of signal.

2. The microarray biochip of claim 1, wherein the intervals between the adhesive units range in 0.1, 1, 10, 100, and larger which the smaller one is preferable.

3. The microarray biochip of claim 1, wherein the-preset height of the adhesive units is at least greater than 10 nm, and ranges in 10, 50, 100, 500 and 1000 nm, which the one smaller than the intervals of the adhesive units is preferable.

4. The microarray biochip of claim 1, wherein the adhesive units are solid and have a cross section formed in a shape of a circle, a rectangle, or a selected geometric shape.

5. The microarray biochip of claim 1, wherein the adhesive units are hollow and have a cross section with an outer shape of a circle, a rectangle, or a selected geometric shape and with an inner shape of a circle, a rectangle, or a selected geometric shape.

6. The microarray biochip of claim 1, wherein the reaction zone is formed on the substrate in an array fashion.

7. The microarray biochip of claim 1, wherein the reaction zone is formed in a shape of a circle, a rectangle, or a selected geometric shape.

8. The microarray biochip of claim 1, wherein the adhesive units are made from materials that have high affinity to biological molecules or biological compatibility, such as gold, silver, copper, nickel or silicon.

9. The microarray biochip of claim 1, wherein the adhesive units are formed on the substrate by employing Micro Electro Mechanical System or nanolithography techniques.

10. The microarray biochip of claim 1, wherein the substrate is glass or other transparent material.

11. A microarray biochip employing biological specificity to immobilize selected biological molecules on substrates as probes to interact with corresponding target of test sample solution to generate signals to facilitate readings and analysis for detection devices, comprising:
a transparent substrate having at least one indented reaction zone which has a fall distance with the adhesive unit; and
a plurality of spaced adhesive units fixedly located in the reaction zone with intervals formed therebetween, each adhesive unit having a preset height and a top surface to form a first adhesive surface and lateral side surfaces to form second adhesive surfaces for the biological probes to immobilize thereon;
wherein the fall distance contains the sample solution and end label products after calorimetric or fluorometric reaction, the second adhesive surfaces providing the probes with the immobilization area for use in optical transmittant detection, the transparent substrate and the intervals allowing optical transmittant type or reflective type of detection device to process reading and analysis of signals.
